(19) United States
(12) Patent Application Publication
(10) Pub. No.: US 2015/0247120 A1
(43) Pub. Date: Sep. 3, 2015

(54) Method for Reprogramming Canine Testicular Cells

(71) Applicant: Chauncey B. Sayre, Huntington Beach, CA (US)

(72) Inventor: Chauncey B. Sayre, Huntington Beach, CA (US)

(21) Appl. No.: 14/634,840

(22) Filed: Mar. 1, 2015

Related U.S. Application Data
(60) Provisional application No. 61/946,548, filed on Feb. 28, 2014.

(51) Int. Cl. C12N 5/076

(52) U.S. Cl. CPC C12N 5/061; C12N 2501/115; C12N 2501/40; C12N 2501/13

(57) ABSTRACT

Canine testes cells can be isolated, cultured, and enriched for spermatogonial stem cells. The canine spermatogonial stem cells can be induced to pluripotency through a relatively simple protocol. These pluripotent cells can be induced to differentiate to various cell- or tissue-types as necessary, and used for therapeutic repair of damaged or diseased cells in canines or to perform veterinary research.

### CLAIM OF BENEFIT TO RELATED APPLICATIONS

This application claims the benefit of U.S. Provisional Application No. 61/946,548, filed Feb. 28, 2014. The contents of application 61/946,548 are incorporated herein by reference.

### FIELD OF INVENTION

The present invention relates to a method of reprogramming canine testicular cells to a less differentiated state.

### BACKGROUND

The use of multipotent and pluripotent cells to generate specific differentiated cells in order to treat canine diseases and injuries, as well as for use in disease modeling and developing an understanding of the effects of various compounds or antigens on cells, should significantly advance veterinary medicine over the next decade. With many dog owners committed to providing their pets with the highest quality of veterinary care available, there is a strong demand for simple but robust stem cell technologies for canines.

Canine induced pluripotent stem cells (IPSCs) have been generated by several groups using fibroblasts transfected with Oct4, Sox2, c-Myc and KLF4 genes (Shimada, Ht. et al., Mol. Reprod. Dev. 77:2 (2010); Lee, A. S. et al., J. Biol. Chem. 286 (37): 32697-32704 (2011); Whitworth, D. J., et al., Stem Cells Dev. 21(12): 2288-2297 (2012)). Recently however, chromosomal instability, including trisomy on multiple chromosomes, has been observed in canine IPSCs (Koh, S. et al., Stem Cells Dev. 22(6): 951-963 (2013)). There are also concerns regarding the fidelity, or lack thereof, of a cell following the introduction of exogenous nucleic acids. Additionally, the fact that some of the exogenous coding sequences, particularly c-Myc, are correlated with various cancers is reason to be cautious, particularly if retroviral vectors are used.

Spermatogonial stem cells (SSCs) represent an appealing substrate for generating multipotent and pluripotent cells. SSCs provide some inherent conceptual advantages with respect to the induction of pluripotency. They represent a highly stable, unipotent cell line that drives spermatogenesis, leading to the production of gametes which, with high fidelity, transmit DNA to offspring that are capable of generating all cell types. In the laboratory, SSCs have been induced to pluripotency in other species, including mice (Kanatsu-Shinohara, M. et al., Cell 119:1001-12 (2004); Ko K. et al., Cell Stem Cell 5:87-96 (2009)) and humans (Conrad, S. et al., Nature 456: 344-349 (2008)).

Accordingly, there exists a need for a simple but effective method of generating canine multipotent or pluripotent stem cells that avoids the potential limitations of IPSC-based approaches.

### SUMMARY OF THE DISCLOSURE

The present disclosure is directed to methods related to canine SSCs that are reprogrammed to a less differentiated state. The induction and isolation of multipotent or pluripotent cells disclosed herein involves a simple, rapid, serum-free process.

In some embodiments, a method of isolating and enriching SSCs from a population of cells from the testes of either a juvenile or adult canine is featured. The method comprises physically dissociating the tissue, contacting the dissociated tissue with collagenase for a length of time, and subsequently contacting it with trypsin for a length of time.

In some embodiments, the canine is administered antibiotics for a period of time prior to the removal of his testes.

In some embodiments, the spermatogonia isolated from the canine testes are cultured in serum-free, defined media on a layer containing a minimal quantity of stromal feeder cells.

In some embodiments, spermatogonia isolated from the canine testes are T75 cm² tissue culture flask with a vented cap, or an equivalent and comparable container that can be used to control gaseous exchange. In some embodiments, the T75 cm² flask is coated with gelatin. In some embodiments, a substrate other than gelatin but possessing similar qualities may coat the T75 cm² flask. In some embodiments, the spermatogonia are cultured in a tissue culture flask or dish other than a T75 cm² flask.

In some embodiments, at least 2.0×10⁷ cells, or 266,667 cells/cm², are initially seeded in the T75 cm² tissue culture flask.

In some embodiments, at least 1.2×10⁷ cells, or 160,000 cells/cm², but less than 2.0×10⁷ cells, or 266,667 cells/cm², are initially seeded in the T75 cm² tissue culture flask.

In some embodiments, at least 8.4×10⁶ cells, or 112,000 cells/cm², but less than 1.2×10⁷ cells, or 160,000 cells/cm², are initially seeded in the T75 cm² tissue culture flask.

In some embodiments, at least 4.0×10⁶, or 53,333 cells/cm², but less than 8.4×10⁶ cells, or 112,000 cells/cm², are initially seeded in the T75 cm² tissue culture flask.

In some embodiments, a high ratio of spermatogonia to stromal cells are transferred to the T75 cm² tissue culture flask, such that one or more spermatogonia associate with, or adhere to, each stromal cell on the surface of the flask.

In some embodiments, spermatogonia isolated from the canine testes are exposed to a culture system that comprises at least one defined medium selected from the group consisting of Dulbecco's Modified Eagle's Medium, Dulbecco's Modified Eagle's Medium/F-12, and StemPro serum replacement supplement.

In some embodiments, spermatogonia isolated from the canine testes are exposed to a culture system that comprises glial cell-derived neurotrophic factor (GDNF), or an equivalent thereto.

In some embodiments, spermatogonia isolated from the canine testes are exposed to a culture system that comprises fibroblast growth factor 2 (FGF2) or basic FGF, or a recombinant form of human FGF that is comparable in function and structure to FGF2 or basic FGF.

In some embodiments, spermatogonia isolated from the canine testes are exposed to a culture system that comprises a glutamine source.

In some embodiments, the spermatogonia isolated from the canine testes are exposed to a culture system that comprises bovine serum albumin and 2-mercaptoethanol.

In some embodiments, the spermatogonia isolated from the canine testes are exposed to a culture system that comprises penicillin and streptomycin.

In some embodiments, the spermatogonia isolated from the canine testes are exposed to a culture system that comprises leukemia inhibitory factor.

In some embodiments, the canine SSCs form hemispherical aggregates with defined borders that resemble embryoid bodies after a period of time under appropriate culture conditions.

In some embodiments, the SSCs that have formed hemispherical aggregates can be isolated. This can be done through simple mechanical scraping, followed by trypsin treatment to divide up the cells. These cells can then be cultured under conditions that promote differentiation. In some embodiments, growth conditions that promote neuronal differentiation can be used.

### DEFINITIONS

Unless defined otherwise, all technical and scientific terms used herein generally have the same meaning as commonly understood by one of ordinary skill in the art to which this invention belongs. Generally, the nomenclature used herein and the laboratory procedures in cell biology, cell culture, molecular genetics, nucleic acid chemistry and hybridization are those well known and commonly employed in the art. In the event of ambiguity concerning the precise meaning of a term, the definitions and explanations contained within the present specification will control.

As used herein, the articles “a” and “an” refer to one or to more than one (i.e., to at least one) of the grammatical object of the article. By way of example, “an element” means one element or more than one element.

As used herein, the term “embryoid body” or “embryoid bodies” refers to one or more three-dimensional multi-cellular aggregates of pluripotent stem cells, capable of generating cell types from the three germ layers.

As used herein, the term “embryonic stem cell,” or “ESC,” refers to pluripotent stem cells derived from the inner cell mass of a blastocyst that maintains the initial, naïve, uncommitted state of pluripotency through self-renewal.

As used herein, the term “epidermal growth factor,” or “EGF,” refers to a growth factor that can stimulate cell growth, proliferation, and differentiation by binding to the epidermal growth factor receptor. Critically, it can also prevent the differentiation of SSCs under some conditions.

As used herein, the term “fibroblast growth factor,” or “FGF,” refers to a large family of growth factors involved in a wide variety of processes, including but not limited to the proliferation and differentiation of a number of cell- and tissue-types in vivo and in vitro, and in some cases, to the inhibition of differentiation.

As used herein, the terms “basic fibroblast growth factor”, “HFGF basic,” “bFGF,” “fibroblast growth factor 2,” and “FGF2,” all refer to a particular member of the fibroblast growth factor family. In stem cell cultures, basic fibroblast growth factor can be used to inhibit differentiation.

As used herein, the term “glial cell-derived neurotrophic factor,” or “GDNF,” refers to a protein encoded by the GDNF gene that promotes the development, maintenance and functioning of neurons, and has been determined to be critical for the self-renewal of SSCs in vivo and to a large degree, in vitro. In the seminiferous tubules, GDNF is secreted by Sertoli cells.

As used herein, the term “leukemia inhibitory factor,” or “LIF,” refers to an interleukin-6 class cytokine that is known to support stem cell self-renewal in a number of mammalian species by inhibiting cell differentiation.

As used herein, the term “multipotent” refers to a stem cell capable of differentiating into multiple cell types, but this differentiation potential is limited compared to that of a pluripotent cell.

As used herein, the term “nude mouse” refers to a mouse lacking a properly functioning thymus, resulting in a greatly reduced quantity of T cells and a dysfunctional immune system, thus allowing it to serve as an ideal subject for immunological studies.

As used herein, the term “paracrine signaling” refers to local, cell to cell communication involving the diffusion of one or more factors capable of influencing the development, or differentiation status of neighboring cells.

As used herein, the term “pluripotent” refers to a stem cell capable of differentiating into cells from any of the three germ layers, namely the ectoderm, endoderm, or mesoderm.

As used herein, the term “reprogramming” refers to the ability to convert a fully or partially differentiated cell to a pluripotent cell.

As used herein, the term “spermatogonium” (plural: “spermatogonia”) refers to an undifferentiated male germline cell in the basement membrane of the seminiferous tubule that is a progenitor of spermatocytes.

As used herein, the term “spermatogonial stem cell” or “SSC” refers to a unipotent, male germline stem cell that helps maintain spermatogenesis.

As used herein, the term “unipotent” refers to a stem cell capable of differentiating into one cell type.

### BRIEF DESCRIPTION OF THE DRAWINGS

FIG. 1 is a simple block flow diagram representing a method of reprogramming cells from the canine testes to a less differentiated state.

FIG. 2 contains two photomicrographs of cells cultured in a media that comprises DMEM/F-12, GDNF, FGF basic, bovine serum albumin (BSA), GlutaMAX, StemPro hESC SFM Growth Supplement and 2-mercaptoethanol. After 72 hours, penicillin-streptomycin was added to the media. The top image displays the cell culture after approximately seven days. Colonies of SSCs associated with fibroblasts on the gelatin-coated surface of the T75 cm² flask are visible. The bottom image, taken at a slightly higher magnification after eight days of culturing also illustrates SSCs associating with fibroblasts.

FIG. 3 contains two photomicrographs illustrating cells derived from canine testes two and a half weeks earlier that had been initially reprogrammed to pluripotency and then cultured for approximately 10 days with neuronal cell media comprising Knockout DMEM/F-12, StemPro Neural Supplement, FGF basic, EGF, and GlutaMAX-I.

### DETAILED DESCRIPTION

The reprogramming of canine spermatogonial stem cells to a less differentiated state through a rapid but simple protocol that employs a serum-free, defined medium would provide significant advantages, offering great promise with respect to the practical, therapeutic treatment of canines as well as various aspects of veterinary research.

### Isolation of Canine Testes Cells

The pathway to generating multipotent or pluripotent, canine spermatogonial stem cells begins with the isolation of testes cells. Male canine reproductive cells can be abstracted through the use of a biopsy tool or they can be retrieved during neutering. The procedure can be performed on either juvenile or adult canines. Younger canines, including neonates, are highly suitable for the protocol disclosed herein, as they possess a higher ratio of spermatogonial stem cells to stromal cells, though one would only neuter a neonatal canine if medically advisable. It should be noted that while the entire protocol for reprogrannmming spennatogonial stem cells (and subsequently differentiating them if desired) has proven very successful with canines, with certain modifications, its use with other species cannot be precluded.

Following abstraction, the testes are decapsulated and the seminiferous tubules are extracted. In the process of extracting the seminiferous tubules, a small fraction of stromal cells is also acquired. These stromal cells, which are typically fibroblasts, serve as ideal feeder cells in the stem cell culture media. Unlike some other feeder cell options, these are neither mitomycin C inactivated nor gamma irradiated, and comprise very vigorous cells. Sertoli, and possibly even Leydig cells, are other somatic cells in the dissociated cell mixture that may act as feeder cells, though it does not appear that they adhere to the gelatin-coated surface of cell culture flasks to any significant extent, unlike the stromal cells.

Cells from the seminiferous tubules can be mechanically separated through the use of an Eppendorf cell separator machine or other devices capable of dissociation. Sequential enzymatic digestions of collagenase IV and trypsin are then used to isolate cells. The now semi-disengaged tissue is placed into a 10 mg/ml collagenase IV solution that includes calcium and magnesium co-factors for approximately 15 minutes, while agitating in a 37° C. water bath. The addition of fetal bovine serum terminates the collagenase IV activity.

The suspension is centrifuged for 6 to 10 minutes at 1,500 to 2,300 rpm (Eppendorf 5280). The supernatant is removed and discarded and then 0.25% trypsin solution is added to the pellet. The pellet is re-suspended and the tube is agitated for up to 10 minutes at approximately 37° C. to further separate the cells. Soybean trypsin inhibitor (Gibco) or fetal bovine serum (Gibco) is added to terminate trypsin activity. The mixture is centrifuged for approximately 8 minutes at about 1,350 to 2,300 rpm and then the supernatant is removed and discarded. The pellet is re-suspended in phosphate buffered saline (PBS) and filtered through a 30 to 40 μm mesh, yielding cells primarily from the seminiferous tubules.

Cell numbers can be counted by taking an aliquot and using it with a hepatocytometer or an automated cell counter, such as a TC20 (Bio-Rad).

### Culturing SSCs and Inducing Pluripotency

Under sterile conditions in a tissue culture hood, the cell filtrate, containing a high ratio of spermatogonia to stromal cells, is placed in 15 to 25 ml serum-free, feeder-free stem cell media and transferred to a gelatin-coated T75 cm² flask (BD BioCoat, Gelatin 75 cm² Flask with vented cap) for approximately 24 to 48 hours at 37° C. and 5% CO₂ atmosphere. It should be noted that depending upon the mass of the starting tissue, the cell filtrate may need to be divided amongst two T75 cm² flasks, however, impressive results have been noted when using a high starting concentration of cells, including more than or equal to 2.0×10⁷ cells, which equates to more than or equal to 266,667 cells/cm² of flask surface area.

The cells are cultured in stem cell media comprising 1×DMEM/F-12 (Gibco), 1×+GlutaMAX (Gibco; GlutaMAX is a stable glutamine dipeptide that produced a final glutamine concentration of approximately 2 mM), approximately 1.8% BSA (Gibco), 1.5 to 18 ng/ml GDNF (Gibco), 8 to 80 ng/ml human fibroblast growth factor basic (Gibco), approximately 0.1 mM 2-mercaptoethanol, and 1× StemPro hESC SFM growth supplement (Gibco). StemPro hESC is a serum-free, feeder-free medium designed for culturing human embryonic stem cell cultures; comparable media from other suppliers can also be used.

The concentrations of GDNF and FGF basic used in the protocol disclosed herein comprised a broad range. The highly successful results observed when using higher concentrations suggest that one or both of these factors may positively affect the transcription of genes associated with pluripotency.

Though sparse in number, the stromal cells are active and serve as feeder cells. Unlike the spermatogonia in the mixture, the stromal cells can attach to the T75 cm² flask's gelatin coating. The remaining cells compete for the nutrition provided by the stromal cells, and begin to adhere to the stromal cells. Multiple spermatogonia adhere to each stromal, feeder cell. Eventually, the spermatogonia adhere to, or associate with, each other as well. The high ratio of spermatogonia to adherent cells results in a three dimensional structure that recapitulates some aspects of early embryonic development and results in differential exposure of cells to morphogens. In a matter of days, hemispheric colonies begin to form.

In an alternative embodiment, one may enrich for SSCs by using a magnetic activated cell sorting system (MACS). MACS functions by using metal bead-conjugated antibodies against cell surface antigens on the SSCs. Integrins, including α6-integrin and β1-integrin, represent potential targets. Other possible targets include the GDNF family receptor α1 (GFRα1) and Thymocyte differentiation antigen-1 (Thy-1), a glycosyl phosphatidylinositol anchored cell surface antigen. Similar enrichment can be performed using fluorescence-activated cell sorting (FACS), whereby fluorescent-conjugated antibodies are first directed against cell surface antigens and then the cell population is sorted by flow cytometry.

Spermatogonial stem cells are unipotent stem cells in their normal niche, or microenvironment, in the seminiferous tubules. Outside of this niche, under the appropriate conditions, it is possible to induce SSCs to pluripotency. Some aspects of the design and effectiveness of the method disclosed herein comprise establishing cell dynamics within the culture system that, in some respects, recapitulate the embryonic environment. The spatial arrangement of cells is of critical importance. This arrangement of cells is set in motion by the use of the gelatin coating on the flask. Coatings other than gelatin may be used, but the properties should be such that adherence is somewhat limited. Only the stromal cells adhered to gelatin, and the quantity of stromal cells used was deliberately limited relative to the spermatogonia. Careful isolation of seminiferous tubules allows the researcher to establish a high ratio of spermatogonia to stromal cells in the T75 cm² tissue culture flask following the transfer of the isolated cell filtrate.

The stromal cells are vigorous but are heavily burdened in this culture, typically having five to ten spermatogonial stem cells associated with each of them. In some cases, there will be small islands of fibroblasts (or other feeder cells) to which multiple SSCs adhere, though the SSCs will outnumber the feeder cells. The SSCs communicate with stromal cells and then later with neighboring spermatogonia via paracrine signaling. The paracrine factors appear capable of inducing pluripotency in neighboring spermatogonial stem cells. This closely parallels the relationship between cells and paracrine factors in early embryos. For example, in the mouse embryo, Nodal, a TGFβ-family protein, is cleaved and activated by two paracrine proteins, Furin and Pace4. Following activation, Nodal helps to maintain pluripotency (Mesnard, D. et al., “The microenvironment patterns the pluripotent mouse epiblast through paracrine Furin and Pace4 proteolytic activities,” Genes Dev. 25(17):1871-1880 (2011)).

Paracrine signaling operates over small distances, limited by diffusion. A morphogen gradient forms and cells proximal to the center of a cellular aggregate may be exposed to a higher concentration of paracrine proteins than more distal cells potentially leading to a change in cell fate, as is the case with the differentiation of the germ layers in early embryogenesis.

Generally it was observed that within approximately two days, clusters of spermatogonia associated with stromal cells began to form hemispherical colonies. In some cases, colonies formed from multiple SSCs associated with small beds comprised of one or more elongated fibroblasts. The three dimensional, hemispherical structures that formed are a trademark of pluripotency, and strongly resemble embryoid bodies.

In one aspect, gelatin-coated T75 cm² flasks are used as opposed to gelatin-coated Petri dishes. The flasks allow for ideal gas exchange under high cell density conditions, creating an environment in which SSCs compete for access to feeder cells. While the benefits of the T75 cm² flasks are apparent, a person having ordinary skill in the art will appreciate that an alternate apparatus can be used for culturing purposes if atmospheric exchange can be limited appropriately.

Successful results have been obtained using LIF-free medium. Nevertheless, after generating hemispherical colonies in culture, LIF can be added to the media if desired. LIF may assist in inhibiting differentiation after the cells have undergone a number of passages.

Through the use of these conditions, cells grow quickly and there is no need for additional enrichment strategies. Induction of multipotency or pluripotency in spermatogonial stem cells occurs very rapidly under the culture conditions described herein.

### Differentiation of Induced Pluripotent Stem Cells

After approximately 7 to 8 days, but in some cases after as few as two days, colonies displaying the hemispherical morphology and defined perimeter associated with pluripotency can be transferred to media containing particular growth factors or other components designed to promote differentiation. In one embodiment, after approximately seven days, hemispherical colonies can be transferred to neuronal stem cell media. This media comprises 1× Knockout DMEM/F-12 (Gibco), a low osmolality medium without L-glutamine or HEPES, 1× StemPro NSC SFM, 1× GlutaMAX-I, 20 μg/ml FGF basic, 10 μg/ml EGF.

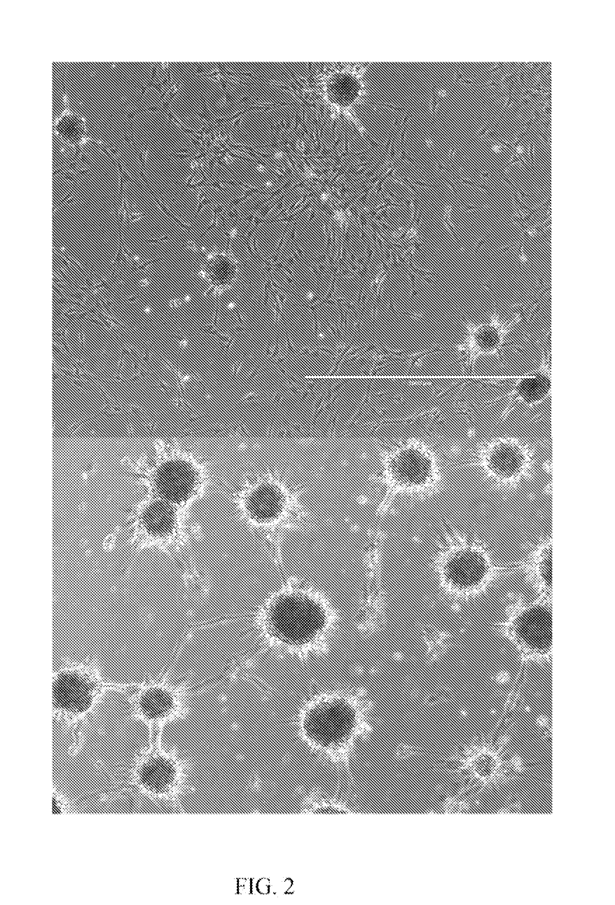

After approximately another 10 days, cells showing different stages of neuronal development and differentiation should be clearly visible, as seen in photomicrographs in FIG. 2 which illustrates the results of Example 2. This neuronal differentiation provides further evidence that SSCs had previously been reprogrammed to pluripotency.

Other factors can certainly be chosen in order to differentiate the induced pluripotent stem cells to other cell- and tissue-lines. The method of generating reprogramming spermatogonial stem cells, as described in this disclosure, represents a valuable tool in therapeutic repair of damaged cells and tissues in vivo. Additionally, the approach could be used to develop organs in vitro, which could subsequently be transplanted.

When following this protocol to induce multipotent or pluripotent spermatogonial stem cells, a stem cell researcher having ordinary skill in the art can readily ascertain the presence of cell cultures bearing the unmistakable morphological characteristics associated with pluripotency, including embryoid body formation. That said, though not strictly necessary, a researcher could perform additional assays to further characterize the results. Transcriptional activity of genes associated with SSCs and pluripotency can be analyzed via RT-PCR. Immunofluourscent techniques can also be used to assess the expression of markers commonly, though not always, associated with pluripotency, such as Oct4, Nanog, Sox2, Lin28, SSEA-1, SSEA-3, SSEA-4, Tra 1-60, Tra 1-81. Polyclonal Oct-4 antibodies can be directed against cultures that have been fixed with paraformaldehyde and then permeabilized with Triton X-100 in PBS. Fluorescent-conjugated secondary antibodies are then used to detect the primary antibody to Oct4.

Alkaline phosphatase activity is present in many undifferentiated cells but declines significantly in differentiated cells. Anecdotal evidence suggests that alkaline phosphatase (AP) activity may not be as pronounced in SSCs as it is in embryonic stem cells, but it can still be assayed in both putative SSCs and differentiated cells, and compared. To assay for AP activity, cultures are fixed in acetone and then stained with napthol AS-BI phosphate/fast red violet solution (Sigma-Aldrich), using techniques familiar to those having ordinary skill in the art. Nude mice can be injected with cells isolated and purified from cell clusters deemed to be pluripotent to monitor for teratoma formation.

Imprinting patterns due to DNA methylation can be analyzed via bisulfate sequencing and compared with the imprinting patterns of cells harvested at earlier stages in the protocol. Importantly, chromosomal stability can be examined, though to date, no evidence suggests that chromosomal instability is a concern with pluripotent canine SSCs, unlike the case with canine IPSCs. To mitigate concerns, copy number status of genomic DNA can be performed by comparative genomic hybridization (CGH). Some of the stromal cells scraped from the membrane of the decapsulated testes previously in the protocol can be used as a reference for normal ploidy in this dual color technique. Microarray-based CGH (Agilent) offers impressive resolution. Fluorescence in situ hybridization (FISH) can be used to confirm specific chromosomal aberrations. Species-specific differences are not uncommon in experimental biology, sometimes necessitating small procedural adjustments. All of the assays mentioned above, however, involve the use of protocols and apparatus well known to those researchers having ordinary skill in the art and detailed protocols are readily available.

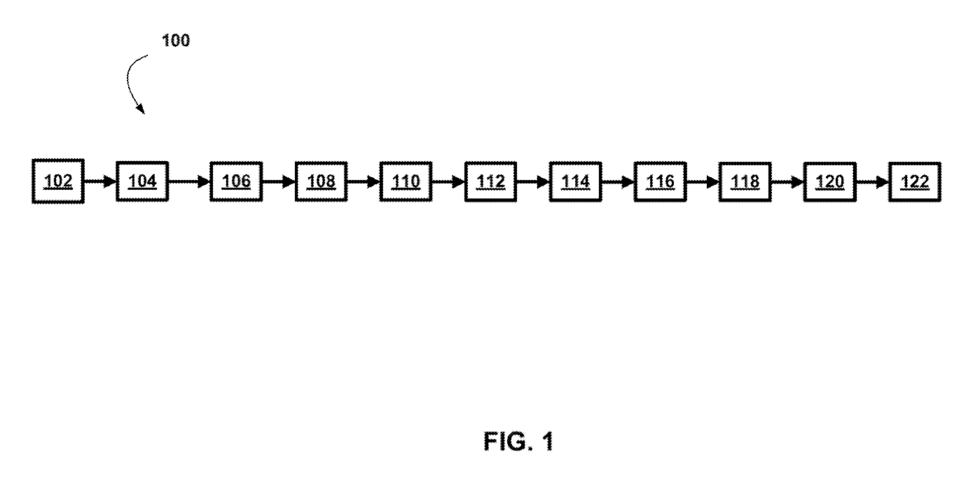

FIG. 1 illustrates a simplified block diagram of a method 100 of reprogramming cells from the canine testes to a less differentiated state. A testicle is surgically removed 102 from the canine. The testicle is subjected to decapsulation and mechanical dissociation 104. Sequential enzymatic digestion 106 with collagenase and trypsin permits the isolation of cells. Cells are resuspended 108 in buffered solution and filtered to remove non-cellular debris. Cell numbers can be counted 110 with a hepatocytometer or an automated cell counter.

Cells are placed in a serum-free, feeder-free stem cell media 112. The media contains a nutrient and growth mixture comprising Dulbecco's Modified Eagle Medium/Nutrient Mixture F-12; a glutamine source, such as GlutaMax; GDNF; FGF 2; StemPro hESC Growth Supplement, and optionally, both bovine serum albumin and 2-mercaptoethanol. Cells are placed 114 in a gelatin-coated T75 cm² flask or equivalent.

Cells are cultured 116 for 24 to 48 hours at 37° C. and 5% CO₂ atmosphere. Media is refreshed. Penicillin-streptomycin is added to media after approximately 72 hours. Visible hemispherical colonies begin to form 118 by the eighth day or sooner. Cells from hemispherical colonies can be isolated 120 through mechanical scraping. These cells can be resuspended in stem cell media that further comprises leukemia-inhibitory factor.

Cells can be resuspended in media that includes one or more factors that promote differentiation 122 to a particular cell type.

### EXAMPLES

The present disclosure is further described by referring to the following experimental examples. These examples help illustrate aspects of the embodiments only, and are not intended to be limiting unless otherwise specified. Thus, the disclosure should in no way be construed as being limited to the following examples, but rather, should be construed to encompass any and all variations which become evident to those having ordinary skill in the art as a result of the teaching provided herein.

### Example 1

Donor: Canine; a 7 to 8 year old male Yorkshire Terrier being neutered.

Mycoplasma is a common infection in canine genital tissue. Pre-treatment of the donor animal is generally preferable and more effective than treating tissue samples and cells post-extraction. Over a 10 day period, the canine donor was pre-treated with antibiotics to prevent mycoplasma contamination. One 22.7 mg tablet of Baytril was given to the canine twice a day for 10 days. In addition, the canine was given approximately 10 mg/kg bodyweight doxycycline per day. The particular antibiotic course may be altered as necessary depending upon the dog's health, breed, and various local considerations.

Following the pre-treatment with antibiotics, testes were obtained from the canine donor through standard veterinary neutering procedures, and the recovered testes were treated. The following protocol was used:

Day 1
1. Obtain a 20 cc sterile syringe. Remove the syringe from its case. Keep the lid to the syringe case sterile.
2. Add 10 ml of saline solution with 100 μl of 100× Penicillin-Streptomycin (Gibco, 10,000 U/ml penicillin, 10,000 μg/ml streptomycin). Carefully place testicle in syringe case without touching the edges. Place lid back onto syringe case.
3. Surround the sample with ice packs
4. Transfer sample to laboratory.
5. Obtain a 50 ml conical centrifuge tube. Use a pipette to add 30 ml of Dulbecco's Phosphate Buffered Saline (DPBS) with Ca²⁺ and Mg²⁺ to the tube and then wash the testicle for 2 to 3 minutes by lightly shaking/inverting the tube.
6. Use forceps to remove the testicle.
7. Spray the testicle with 70% alcohol.
8. Place the testicle in a conical tube with fresh DPBS solution. Invert the tube 3 to 4 times before removing the testicle with a new pair of forceps.
9. Transfer testicle to Petri dish with PBS (to keep it moist). Decapsulate the testicle, scrape out the seminiferous tubules and cut them into small fragments.
10. Place the fragments into a gentleMACS test tube. Add 3 ml of Hank's Balanced Salt Solution (Gibco) in gentleMACs tube.
11. Use a gentleMACS Dissociator to mechanically dissociate the testicle fragments. “Brain setting” is the preferred mode. Transfer the sample to a 50 ml conical centrifuge tube.
12. Place the tube into the centrifuge for 6 to 8 minutes at 1,350 to 2,300 rpm.
13. Remove the tube from the centrifuge. Use a pipette to carefully remove the supernatant. Discard the supernatant.
14. Dilute 1 g of collagenase IV by adding 100 ml of Dulbecco's Phosphate Buffered Saline (DPBS) with Ca(+) and Mg (+), yielding a concentration of 10 mg/ml collagenase IV. The concentration can be adjusted accordingly based on the age and condition of canine.
15. Filter the collagenase solution with a 0.1 μm filter.
16. Add 5 ml of diluted collagenase to the sample.
17. Place the tube in a water bath at 37° C. for approximately 8 minutes, swirling the tube every 2 to 3 minutes. The sample's exposure to collagenase IV can be increased if necessary, but should not exceed 15 minutes.
18. Filter fetal bovine serum (FBS) solution with a 0.1 μm filter.
19. Add 10 ml of FBS to stop the reaction of collagenase IV. Centrifuge for 8 to 10 minutes at 1,500 to 2,300 rpm.
20. Remove the tube from the centrifuge. Use a pipette to carefully remove the supernatant. Discard the supernatant.
21. Add 10 ml of 0.25% trypsin into the tube.
22. Place the tube in the 37° C. water bath. Agitate the tube every 2 minutes for up to 10 minutes.
23. Add 10 ml of FBS to neutralize trypsin.
24. Place the tube into the centrifuge for 8 minutes at 1,350 to 2,300 rpm.
25. Remove the tube from the centrifuge. Use a pipette to carefully remove the supernatant. Discard the supernatant.
26. Add 15 to 25 ml stem cell media (see instructions below) to the pellet. Use a pipette to mix the pellet and solution together.
27. Place a 40 μm mesh on top of a 50 ml tube. Use a pipette to transfer the solution onto the 40 μm mesh over the 50 ml tube.
28. A small aliquot of the filtered solution can be used to count cells in a hemocytometer or a TC20 automated cell counter (Bio-Rad).
29. Obtain a gelatin-coated T75 cm² flask and transfer the filtered solution by pipette into the flask using sterile conditions in a tissue culture hood.
30. Place in incubator at 37° C. with an atmosphere of 5% CO₂ in air.

To make the stem cell media, using sterile conditions in a tissue culture hood:
1. Obtain 500 ml of Dulbecco's Modified Eagle Medium/Nutrient Mixture F-12 (DMEM/F-12, Gibco) supplemented with GlutaMAX (Gibco) and BSA (Bovine Serum Albumin, Gibco).
2. Add 80 μl of 100 μg/ml Recombinant Human GDNF (Gibco) lyophilized to the DMEM/F-12+ GlutaMAX, BSA bottle.
3. Add 40 ml BSA 25% (use an entire bottle) to the DMEM/F-12 bottle.
4. Add 10 ml StemPro hESC SFM Growth supplement (Gibco) (entire bottle supplement) to the DMEM/F-12 bottle.
5. Add 400 μl of 100 μg/ml of recombinant human fibroblast growth factor basic (Rec HU FGF Basic, Gibco) to the DMEM/F-12 bottle.
6. Add 909 μl of 55 mM 2-mercaptoethanol (Gibco) to the DMEM/F-12 bottle. Note: 2-mercaptoethanol must be added to the media daily and changed according to volume.
7. After approximately 72 hours growth, penicillin-streptomycin is added to the media. This is accomplished by adding 5 ml of penicillin-streptomycin to the DMEM/F-12 bottle.

Day 2 or later until approximately Day 8 or later
1. Aliquot 15 to 25 ml of media into a conical tube. Put in water bath at 37° C. for 10 minutes.
2. To change the media, remove the old media from flask with a pipette and discard it.
3. Add 15 to 25 ml of fresh media to the flask.
4. Return flask to incubator.

FIG. 2 is a pair of photomicrographs showing cells derived from canine testes that have been cultured according to the protocol disclosed herein. The top image displays cells that have been cultured for seven days. Five large cellular bodies displaying hemispherical shape with defined borders are visible and this morphology is clearly consistent with pluripotency. The more linear cellular structures are fibroblasts. The bottom image, at slightly higher magnification, represents cells cultured for approximately eight days of incubation per the pluripotency protocol outlined herein. Over a dozen large cellular bodies are evident. The hemispherical shape and defined borders are a hallmark of pluripotent morphology.

### Example 2

### Subsequent Differentiation of Reprogrammed Spermatogonial Stem Cells

1. After Day 8 of the procedure shown above in Example 1, colonies demonstrate stem-like morphology, as evident when examined by microscope at 40 to 200× magnification.
2. The cells are subsequently passaged to a neuronal stem culture. The neuronal stem cell culture is made under sterile conditions in a tissue culture hood, and comprises:
   500 ml Knockout DMEM/F-12 (Gibco), a low osmolality medium without L-glutamine or HEPES;
   10 ml of StemPro Neural Supplement (Gibco), a serum-free growth formula designed to support the growth of neuronal stem cell lines;
   500 μl of 20 μg/ml recombinant human FGF basic (Gibco);
   500 μl 10 μg/ml recombinant human EGF (Gibco), and
   5 ml GlutaMAX-I (Gibco) a dipeptide glutamine source.
3. T75 cm² flask is incubated at 37° C.
4. After approximately 10 more days, the cells are analyzed under microscope.

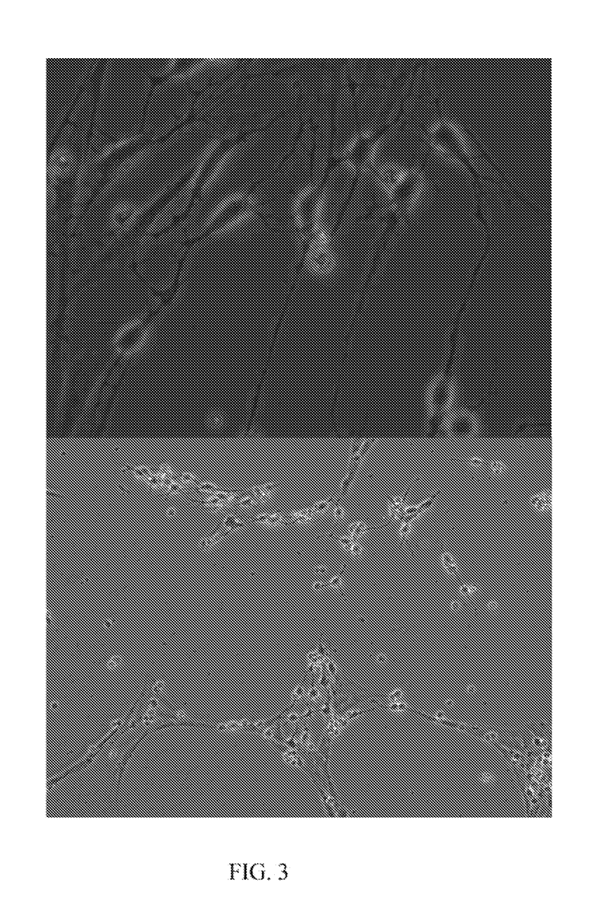

FIG. 3 is a pair of images showing the results of a photomicrograph of cells derived from canine testes two and a half weeks earlier, that had been reprogrammed to pluripotency (as evidenced previously by hemispheric colonies) and then cultured for approximately 10 days with neuronal cell media as described above. The morphology of the cells indicates that the cells have undergone neuronal differentiation, as elongated nerve cell processes are extending from cell bodies.

The disclosure has been described in terms of particular embodiments found or proposed to comprise specific modes for the practice of the disclosure. Various modifications and variations of the described invention may be evident to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the relevant fields are intended to be within the scope of the following claims.

1. A method for reprogramming canine spermatogonial stem cells to a less differentiated state in canine spermatogonial stem cells, comprising:
(a) isolating testes cells from a canine, wherein the isolated testes cells comprise a high ratio of spermatogonial stem cells to stromal cells;
(b) transferring the isolated testes cells to a cell culture growth medium in a container coated with a surface substrate;
(c) culturing the transferred testes cells in the cell culture growth medium until hemispherical colonies are observed on the surface of the container, and
(d) isolating the hemispherical colonies from the surface of the container.

2. The method of claim 1, wherein isolating testes cells comprises: surgically removing a testicle; decapsulating the testicle; mechanically dissociating tissue from said testicle; enzymatically digesting the mechanically dissociated tissue with collagenase and trypsin, and filtering the substrate through a fine mesh to remove larger particles.

3. The method of claim 1, wherein the surface substrate is gelatin or another substrate with similar adherent properties.

4. The method of claim 1, wherein the culturing of testes cells in the container comprises culturing testes cells at an initial density of at least 266,667 testes cells/cm².

5. The method of claim 1, wherein the culturing of testes cells in the container comprises culturing testes cells at an initial density of at least 160,000 testes cells/cm².

6. The method of claim 1, wherein the culturing of testes cells in the container comprises culturing testes cells at an initial density of at least 112,000 testes cells/cm².

7. The method of claim 1, wherein the culturing of testes cells in the container comprises culturing testes cells at an initial density of at least 53,333 testes cells/cm².

8. The method of claim 1, wherein the cell culture growth medium is capable of supporting pluripotent cells in an undifferentiated and pluripotent state.

9. The method of claim 1, wherein the cell culture growth medium comprises essential nutrients, a source of glutamine, glial cell-derived neurotrophic factor (GDNF), fibroblast growth factor basic (FGF 2) and a serum-free, feeder free medium.

10. The method of claim 9, wherein the concentration of glutamine is approximately 2 mM, the concentration of GDNF is between 1.5 ng/ml and 18 ng/ml, and the concentration of FGF 2 is between 8 ng/ml and 80 ng/ml.

11. The method of claim 1, wherein the cell culture growth medium further comprises bovine serum albumin (BSA) and 2-mercaptoethanol.

12. The method of claim 8, wherein leukemia-inhibitory factor (LIF) is added to the cell culture media after hemispherical colonies begin to form.

13. The method of claim 1, further comprising:
(a) isolating hemispherical colonies by mechanical scraping;
(b) treating the isolated colonies with trypsin;
(c) culturing the trypsin-treated colonies in a growth medium that comprises at least one differentiation factor, and
(d) examining the growth medium for changes to cell morphology.

14. The method of claim 13, wherein the growth medium comprises one or more factors that promote neuronal differentiation.
